(19) United States
(12) Patent Application Publication
(10) Pub. No.: US 2025/0213706 A1
(43) Pub. Date: Jul. 3, 2025

(54) MULTIFUNCTIONAL PROTEOLYSIS-TARGETING CHIMERA CONJUGATES

(71) Applicant: BIOXEL INC., Cambridge, MA (US)

(72) Inventors: Patrick R. GLEASON, Millis, MA (US); Elazer R. EDELMAN, Brookline, MA (US)

(73) Assignee: BIOXEL INC., Cambridge, MA (US)

(21) Appl. No.: 18/917,842

(22) Filed: Oct. 16, 2024

Related U.S. Application Data
(60) Provisional application No. 63/590,946, filed on Oct. 17, 2023.

(51) Int. Cl. A61K 47/64; A61K 47/54; A61K 47/55; A61K 47/60; A61P 35/00

(52) U.S. Cl. CPC A61K 47/64; A61K 47/545; A61K 47/55; A61K 47/60; A61P 35/00

(57) ABSTRACT

The present invention relates to various molecular constructs having at least one cellular-targeting element, one protein-targeting element, and one ubiquitin ligase recruitment element, wherein these elements are conjugated in a manner that facilitates cellular uptake and targeted degradation of specific proteins within cells. The present invention further relates to multifunctional proteolysis-targeting chimera conjugates and uses thereof for the treatment of diseases or disorders such as immune and inflammatory diseases, infectious diseases, cardiovascular diseases, endocrine disorders, and various cancers that are otherwise resistant to traditional therapeutic regimens.

### CROSS-REFERENCE TO RELATED APPLICATIONS

This application claims the benefit of U.S. Provisional Applications No. 63/590,946, filed Oct. 17, 2023, which is hereby incorporated by reference.

### SEQUENCE LISTING STATEMENT

A Sequence Listing conforming to the rules of WIPO Standard ST.26 is hereby incorporated by reference. The Sequence Listing has been filed as an electronic document encoded as XML in UTF-8 text. The electronic document, created on Oct. 10, 2023, is entitled “P-628393-USP_ST26.xml” and is 20,950 bytes in size.

### FIELD OF THE INVENTION

The present invention relates to various molecular constructs having at least one cellular-targeting element, one protein-targeting element, and one ubiquitin ligase recruitment element, wherein these elements are conjugated in a manner that facilitates cellular uptake and targeted degradation of specific proteins within cells. The present invention further relates to multifunctional proteolysis-targeting chimera conjugates and uses thereof for the treatment of diseases or disorders such as immune and inflammatory diseases, infectious diseases, cardiovascular diseases, endocrine disorders, and various cancers that are otherwise resistant to traditional therapeutic regimens.

### BACKGROUND OF THE INVENTION

Targeting intracellular proteins implicated in the pathogenesis of a variety of diseases, including the leading causes of mortality and morbidity such as cancers, cardiovascular diseases, and even infectious diseases, has been a long-standing challenge in the field of drug discovery and development. Many of these proteins have been termed “undruggable” due to their lack of traditional small-molecule binding sites or the issues related to drug delivery and specificity. This problem is further exacerbated when these target proteins are overexpressed or mutated in diseased cells, contributing to disease progression and therapeutic resistance.

One promising approach to circumvent these challenges is a class of bifunctional molecules capable of recruiting the cellular ubiquitin-proteasome system to selectively degrade target proteins known as Proteolysis-Targeting Chimeras (PROTACs). Traditional PROTACs comprise two distinct moieties: a protein-targeting moiety and an effector moiety that recruits a ubiquitin ligase to the protein of interest for selective degradation. Several of these molecules have shown promise in preclinical pharmacological studies for their ability to degrade proteins that are typically difficult to target with conventional small molecules.

Yet, despite these advances, delivering PROTACs to diseased cells remains a challenge. The non-specific distribution of the PROTAC during circulation in the body can lead to off-target effects and decreased therapeutic efficacy. In addition, many potential target proteins are located inside cells, further complicating the delivery of these therapeutic agents.

The present invention overcomes these issues by incorporating additional functionality to the PROTAC molecule with cellular targeting peptides. These peptides are designed to bind to specific receptors overexpressed on the surface of diseased cells, guiding the delivery of attached therapeutics specifically to the cell of interest. This targeted delivery approach can increase the therapeutic index by enhancing efficacy and reducing systemic toxicity.

### SUMMARY OF THE INVENTION

In one aspect, the present invention provides a compound comprising
(a) two or more active moieties,
(b) a central molecule, and
(c) two or more linking groups;
wherein each of the two or more active moieties is independently linked to the central molecule through a linking group;
wherein the active moieties comprise a variety of functions that target extracellular elements, intracellular elements, and intracellular processes;
wherein the active moieties are synergistic; and
wherein the active moieties have a molecular weight of from 100 Da to 10⁶ Da.

In some embodiments of the compound of the invention, the compound is represented by the structure of Formula (I)

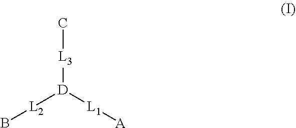

wherein
   A is a cellular targeting moiety;
   B is a protein-binding moiety;
   C is a ubiquitin ligase recruitment moiety;
   D is a central linking moiety; and
   each of L1, L2, and L3 is independently a linking group.

In another aspect, the present invention provides a method for treating a disease or disorder characterized by the overexpression of a specific intracellular protein, comprising administering to a subject in need thereof a therapeutically effective amount of the compound of the invention as described anywhere herein. In some embodiments, the intracellular protein is a KRAS mutant. In some embodiments of the method of the invention, the disease or disorder is selected from immune and inflammatory diseases, infectious diseases, cardiovascular diseases, endocrine disorders, and cancers. In some embodiments, the disease or disorder is resistant to traditional therapeutic regimens.

### BRIEF DESCRIPTION OF THE DRAWINGS

The following drawing forms part of the present specification and are included to further demonstrate certain aspects of the present disclosure, the inventions of which can be better understood by reference to one or more of these drawings in combination with the detailed description of specific embodiments presented herein.

FIG. 1 depicts an embodiment of a therapeutic compound comprising an iRGD tumor homing peptide, a small molecule E3 ligase (VHL) recruiter, and a K-Ras(G12D) binder provided by the present disclosure.

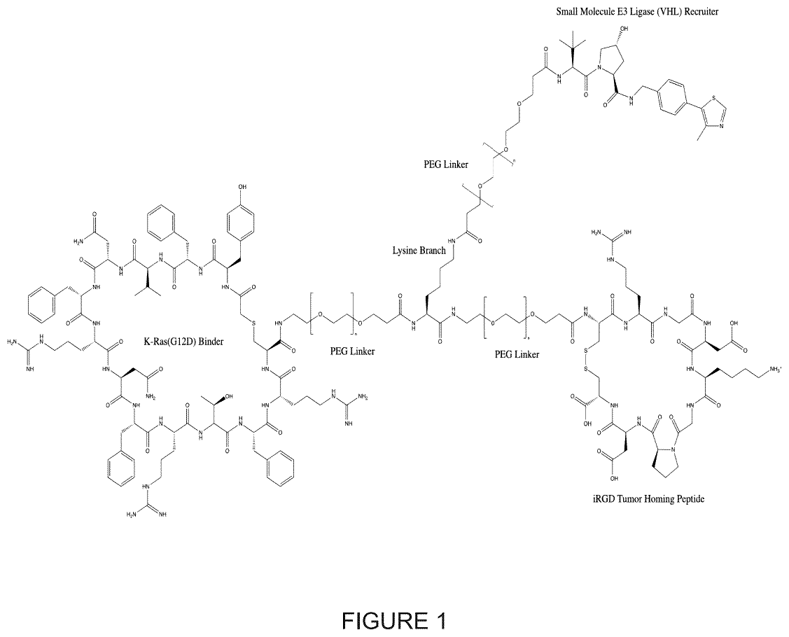

FIG. 2 depicts an embodiment of a therapeutic compound comprising an active cellular targeting moiety containing the cyclic peptide iRGD motif comprising the nine amino acid sequence Cys-Arg-Gly-Asp-Lys-Gly-Pro-Asp-Cys (SEQ ID NO: 1), a small molecule E3 ligase (VHL) recruiter, and a small molecule protein recruiter, which is an inhibitor of a bromodomain (bromodomain and extra-terminal domain (BET) family of proteins consists of four conserved members (Brd2, Brd3, Brd4, and Brdt)), wherein the BRD2, BRD3, and BRD4 inhibitor is OTX015, provided by the present disclosure.

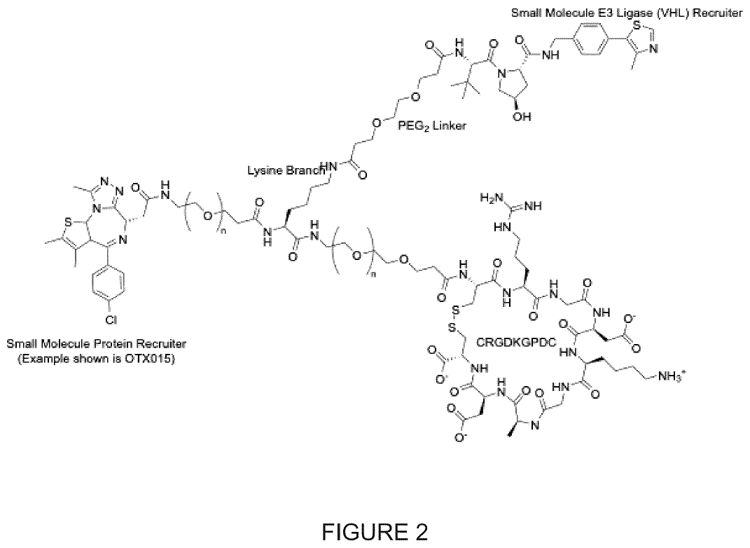

FIG. 3 depicts an embodiment of a therapeutic compound (BXL_2023.001) comprising an iRGD tumor homing peptide, a small molecule E3 ligase (VHL) recruiter, and a K-Ras(G12D) binder provided by the present disclosure.

FIG. 4 is a plot of cell viability as a function of drug concentration for BXL_2023.001 (and depicted in FIG. 3) and showing specificity for pancreatic cancer cell line ASPC-1.

It will be appreciated that for simplicity and clarity of illustration, elements shown in the figure have not necessarily been drawn to scale. For example, the dimensions of some of the elements may be exaggerated relative to other elements for clarity. Further, where considered appropriate, reference numerals may be repeated in the figure to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention relates to novel compounds designed for targeted intracellular protein degradation. The compounds are characterized by their unique structures and multifunctionalities, which allow them to target specific cellular elements and processes.

### Structural Components and Features

(1) The compound comprises:
(i) two or more active moieties,
(ii) a central molecule, and
(iii) two or more linking groups.

(2) Each active moiety is independently linked to the central molecule through a linking group.

(3) The active moieties are designed to target extracellular elements, intracellular elements, and intracellular processes. They possess a molecular weight ranging from 100 Da to 10⁶ Da.

(4) The active moieties are synergistic in nature, enhancing the overall efficacy of the compound.

In some embodiments, the compounds are represented by the structure of Formula (I),

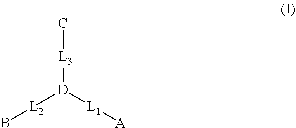

wherein
A represents a cellular targeting moiety,
B signifies a protein-binding moiety,
C denotes a ubiquitin ligase recruitment moiety,
D is a central linking moiety, and
L1, L2, and L3 are each independently linking groups.

### Specific Embodiments

(i) The cellular targeting moiety (A) can be a linear or cyclic peptide. Specific sequences of this moiety can target cellular receptors like αVβ3, αVβ5, and neuropilin-1, which are often overexpressed in tumor cells.

(ii) The protein-binding moiety (B) can also be a linear or cyclic peptide. One embodiment is a cyclic peptide with a specific sequence.

(iii) The ubiquitin ligase recruitment moiety (C) is a small molecule that can specifically target domains of the E3 ligase complex, such as CRBN, VHL, MDM2, and CIAP.

(iv) The central linking moiety (D) can be an amino acid or its derivative, such as lysine, azido lysine, and cysteine.

(v) The linking groups L1, L2, and L3 can be the same or different. They can be polyethylene glycol (PEG), amino acids, hydrocarbons with terminal carboxylic acid and amine groups, or combinations thereof.

### Applications

(i) The compound of the invention is capable of degrading target proteins in cells, with one specific target being the KRAS mutant.

(ii) The invention also encompasses a method for treating diseases or disorders characterized by the overexpression of specific intracellular proteins. This method involves administering the compound to a subject.

(iii) The diseases or disorders that can be treated include, but are not limited to, immune and inflammatory diseases, infectious diseases, cardiovascular diseases, endocrine disorders, and various cancers, especially those resistant to traditional therapeutic regimens, such as pancreatic cancer.

(iv) The present invention, with its multifunctional compound, offers a promising approach to targeted protein degradation, potentially revolutionizing the treatment of various diseases and disorders.

In another aspect, the present invention provides a compound comprising
(a) two or more active moieties,
(b) a central molecule, and
(c) two or more linking groups;
wherein each of the two or more active moieties is independently linked to the central molecule through a linking group;
wherein the active moieties comprise a variety of functions that target extracellular elements, intracellular elements, and intracellular processes; and
wherein the active moieties are synergistic.

In some embodiments of the compound of the invention, the active moieties have a molecular weight of from 100 Da to 10⁶ Da.

In another aspect, the present invention provides a compound comprising
(a) two or more active moieties,
(b) a central molecule, and
(c) two or more linking groups;
wherein each of the two or more active moieties is independently linked to the central molecule through a linking group;
wherein the active moieties comprise a variety of functions that target extracellular elements, intracellular elements, and intracellular processes;
wherein the active moieties are synergistic; and
wherein the active moieties have a molecular weight of from 100 Da to 10⁶ Da.

In one aspect, the present invention provides a compound comprising
(a) two or more active moieties,
(b) a central molecule, and
(c) two or more linking groups;
wherein each of the two or more active moieties is independently linked to the central molecule through a linking group;
wherein the active moieties comprise a variety of functions that target extracellular elements, intracellular elements, and intracellular processes;
wherein the active moieties are synergistic;
wherein the active moieties have a molecular weight of from 100 Da to 10⁶ Da; and
wherein the linking groups have a length of from 1 angstrom to 10 nanometers.

In some embodiments of the compound of the invention, the active moieties comprise a tumor homing peptide, a protein binding peptide, and an E3 ligase recruiter.

As used herein, the “tumor homing peptide” is any peptide capable of targeting tumor cells. The “protein binding peptide” refers to any peptide that is capable of binding to a specific protein. The “E3 ligase recruiter” refers to any molecule capable of recruiting an E3 ligase.

In some embodiments, the tumor homing peptide, the protein binding peptide, and the E3 ligase recruiter are each independently linked to the central molecule through a linking group.

In some embodiments, the central molecule is an amino acid or a derivative thereof.

In some embodiments, the two or more linking groups are identical. In other embodiments, the two or more linking groups are not identical.

In some embodiments, the linking groups are selected from the group consisting of a bond, an amino acid, a polyethylene glycol (PEG), and a combination thereof.

In some embodiments, the compound of the invention comprises a tumor homing peptide, a protein binding peptide, and an E3 ligase recruiter, each independently being connected to a central molecule through a linking group, wherein the linking group is selected from the group consisting of a bond, an amino acid, a polyethylene glycol (PEG), and a combination thereof.

In some embodiments, the tumor homing peptide, the protein binding peptide, and the E3 ligase recruiter are arranged in any order or configuration.

In some embodiments, the compound of the invention is a multifunctional proteolysis-targeting chimera (mPROTAC) conjugate.

In yet another aspect, the compound of the invention is represented by the structure of Formula (I)

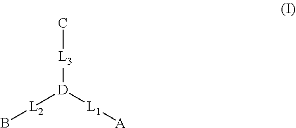

wherein
   A is a cellular targeting moiety;
   B is a protein-binding moiety;
   C is a ubiquitin ligase recruitment moiety;
   D is a central linking moiety; and
   each of L1, L2, and L3 is independently a linking group.

In some embodiments, the active cellular targeting moiety (A) is a linear peptide or a cyclic peptide.

In some embodiments, the active cellular targeting moiety (A) targets αVβ3 and αVβ5 integrin receptors, which may be overexpressed on tumor cells followed by neuropilin-1 receptor-mediated cellular uptake.

In some embodiments, the peptide sequence of the active cellular targeting moiety (A) contains the RGD peptide motif comprising the three amino acids Arg-Gly-Asp. In some embodiments, the peptide sequence comprises the structure of Formula (A1) at neutral pH,

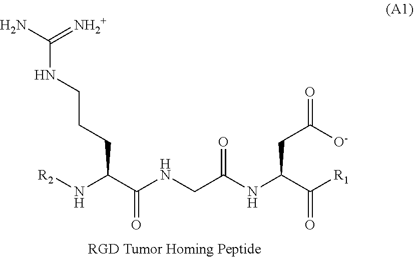

wherein
R₁ is a carboxylate OH or a carboxamide NH₂ and
R₂ is the remaining structure of the compound of Formula (I).

In some embodiments, the peptide sequence of the active cellular targeting moiety (A) contains the cyclic peptide iRGD motif comprising the nine amino acid sequence Cys-Arg-Gly-Asp-Lys-Gly-Pro-Asp-Cys (SEQ ID NO: 1).

In some embodiments, the peptide sequence of the active cellular targeting moiety (A) contains the cyclic peptide iRGD motif comprising the nine amino acid sequence which is a Lys to Arg variant of SEQ ID NO: 1 at position 5 (Cys-Arg-Gly-Asp-Arg-Gly-Pro-Asp-Cys).

In some embodiments, the peptide sequence of the active cellular targeting moiety (A) contains the cyclic peptide iRGD motif comprising the nine amino acid sequence which is an Asp to Glu variant of SEQ ID NO: 1 at position 8 (Cys-Arg-Gly-Asp-Lys-Gly-Pro-Glu-Cys).

In some embodiments, the peptide sequence of the active cellular targeting moiety (A) contains the cyclic peptide iRGD motif comprising the nine amino acid sequence which is a Lys to Arg variant at position 5 and Asp to Glu variant at position 8 of SEQ ID NO: 1 (Cys-Arg-Gly-Asp-Arg-Gly-Pro-Glu-Cys).

In some embodiments of the compound of the invention, the peptide is cyclized via a disulfide bond between cysteine at residue position 1 and cysteine at residue position 9.

In some embodiments, the peptide sequence comprises the structure of Formula (A2) at neutral pH

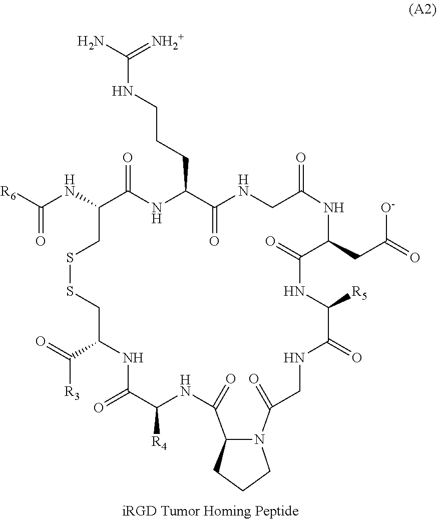

wherein
R₃ is a carboxylate OH or a carboxamide NH₂;
R₄ is an aspartic acid or a derivative thereof, or a glutamic acid or a derivative thereof;
R₅ is a lysine or a derivative thereof, or an arginine or a derivative thereof; and
R₆ is the remaining structure of the compound of Formula (I).

In some embodiments, the peptide sequence of the active cellular targeting moiety (A) contains the NGR motif comprising the three amino acids Asn-Gly-Arg. In some embodiments, the peptide sequence comprises the structure of Formula (A3) at neutral pH

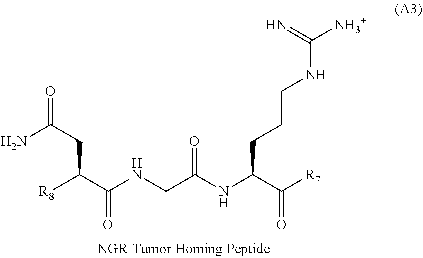

wherein
R₇ is a carboxylate OH or a carboxamide NH₂; and
R₈ is the remaining structure of the compound of Formula (I).

In some embodiments, the peptide sequence contains the NGR motif comprising the five amino acids Cys-Asn-Gly-Arg-Cys (SEQ ID NO: 2). In some embodiments, the peptide is cyclized with a disulfide bond between cysteine at residue position 1 and cysteine at residue position 5. In some embodiments, the peptide sequence comprises the structure of Formula (A4) at neutral pH

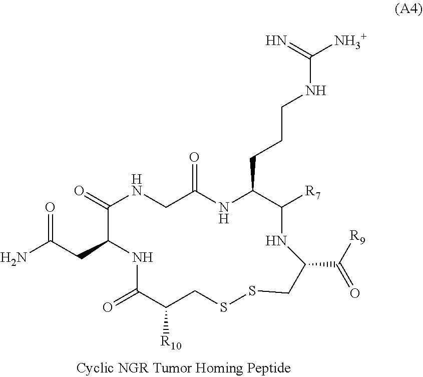

wherein
R₉ is a carboxylate OH or a carboxamide NH₂; and
R₁₀ is the remaining structure of the compound of Formula (I).

In some embodiments, the active cellular targeting moiety targets the extracellular enzyme Aminopeptidase N (CD13).

In some embodiments, the peptide sequence of the active cellular targeting moiety (A) contains the cyclic peptide comprising the nine amino acid sequence Cys-Gly-Asn-Lys-Arg-Thr-Arg-Gly-Cys (SEQ ID NO: 3). In some embodiments, the peptide is cyclized with a disulfide bond between cysteine at residue position 1 and cysteine at residue position 9. In some embodiments, the peptide sequence comprises the structure of Formula (A5) at neutral pH

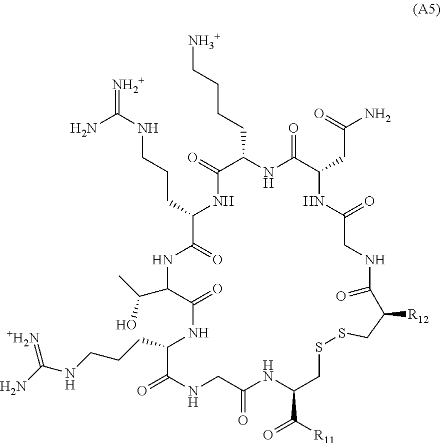

wherein
R₁₁ is a carboxylate OH or a carboxamide NH₂; and
R₁₂ is the remaining structure of the compound of Formula (I).

In some embodiments, the active cellular targeting moiety targets the cellular receptor p32/gC1qR.

In some embodiments of the compound of the invention, the peptide sequence of the active cellular targeting moiety (A) comprises the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 4).

In some embodiments, the peptide sequence comprises the structure of Formula (A6) at neutral pH

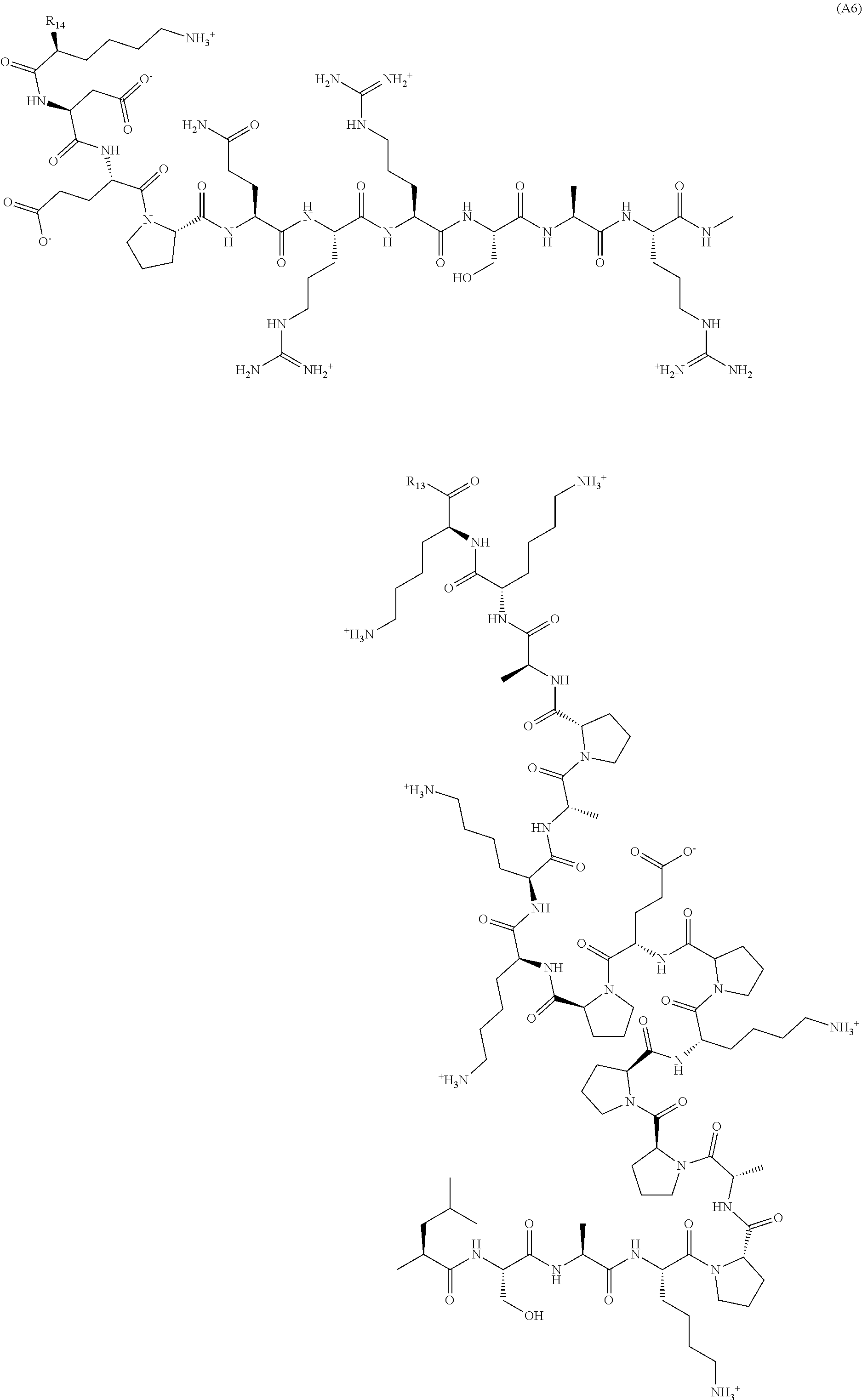

wherein
R₁₃ is a carboxylate OH or a carboxamide NH₂; and
R₁₄ is the remaining structure of the compound of Formula (I).

In some embodiments, the active cellular targeting moiety targets the nucleolin cellular receptor.

In some embodiments, the peptide sequence of the active cellular targeting moiety (A) comprises the amino acids Cys-Arg-Glu-Lys-Ala (SEQ ID NO: 5). In some embodiments, the peptide sequence comprises the structure of Formula (A7) at neutral pH

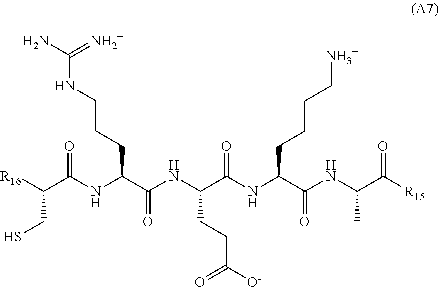

wherein
R₁₅ is a carboxylate OH or a carboxamide NH₂; and
R₁₆ is the remaining structure of the compound of Formula (I).

In some embodiments, the active cellular targeting moiety targets Fibrin-fibronectin complexes.

In some embodiments, the peptide sequence of the active cellular targeting moiety (A) comprises the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 6).

In some embodiments, the peptide sequence comprises the structure of Formula (A8) at neutral pH

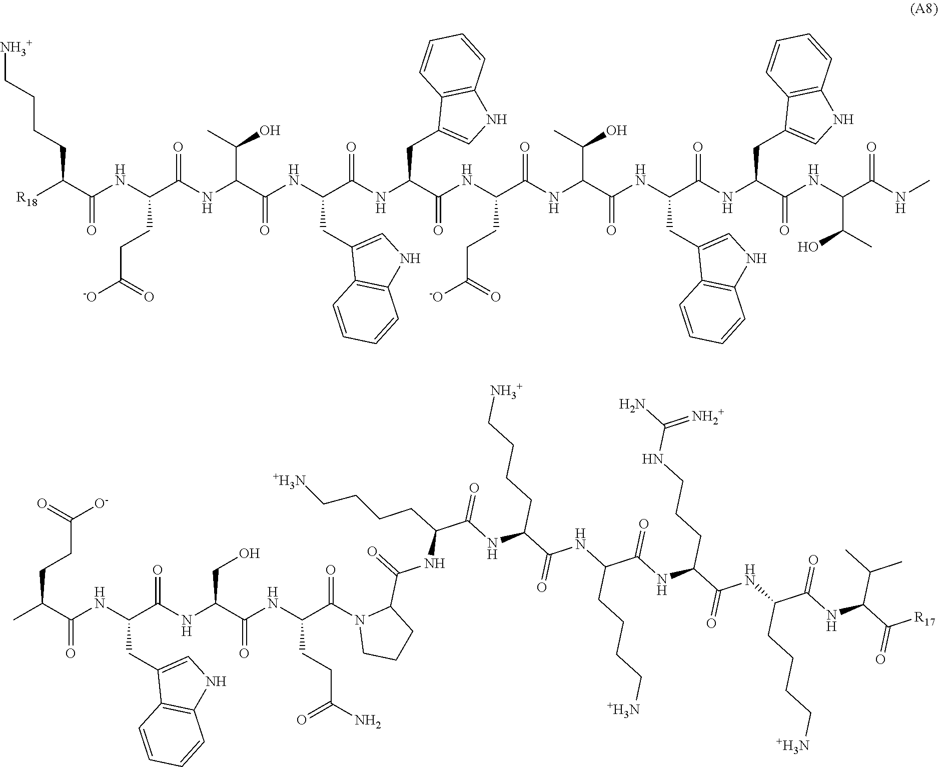

wherein
R₁₇ is a carboxylate OH or a carboxamide NH₂; and
R₁₈ is the remaining structure of the compound of Formula (I).

In some embodiments, the peptide sequence of the active cellular targeting moiety (A) comprises the amino acids Lys-Glu-Thr-Trp-Trp-Glu-Thr-Trp-Trp-Thr-Glu-Trp-Ser-Gln-Pro-Lys-Lys-Lys-Arg-Lys-Val-cysteamine (SEQ ID NO: 7).

In some embodiments, the peptide sequence comprises the structure of Formula (A9) at neutral pH

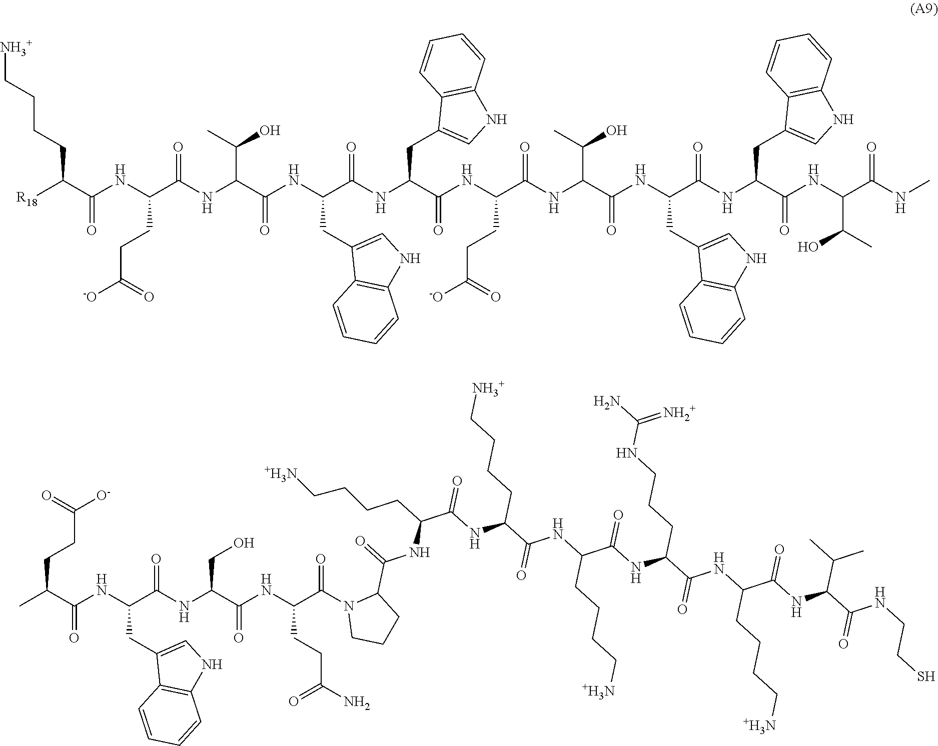

wherein R₁₉ is the remaining structure of the compound of Formula (I).

In some embodiments, the active cellular targeting moiety targets the cellular receptor GRP78.

In some embodiments, the protein-binding moiety (B) is a linear peptide or a cyclic peptide.

In some embodiments, the protein-binding moiety (B) is a cyclic peptide with the sequence dTyr-Phe-Val-Asn-Phe-Arg-Asn-Phe-Arg-Thr-Phe-Arg-Cys-Gly (SEQ ID NO: 8), wherein dTyr represents the D-isomer of tyrosine.

In some embodiments, the peptide is cyclized with a thioether bond between dTyr at residue position 1 and residue position 13.

In some embodiments, the peptide sequence comprises the structure of Formula (B1) at neutral pH

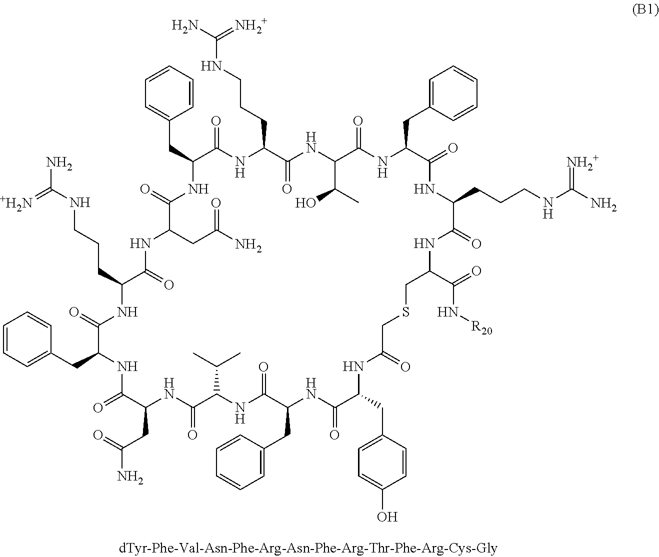

In some embodiments, the ubiquitin ligase recruitment moiety (C) is a small molecule with specificity for the CRBN domain of the E3 ligase complex.

In some embodiments, the ubiquitin ligase recruitment moiety (C) is 2-(2,6-dioxopiperidin-3-yl)-4-hydroxyisoindoline-1,3-dione

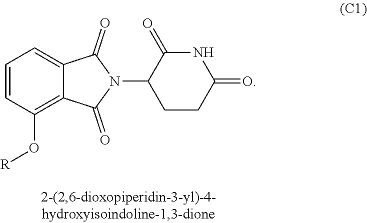

In some embodiments, the ubiquitin ligase recruitment moiety (C) is 3-(4-amino-1-oxoisoindolin-2-yl)piperidine-2,6-dione

In some embodiments, the ubiquitin ligase recruitment moiety (C) is 4-amino-2-(2,6-dioxopiperidin-3-yl)isoindoline-1,3-dione

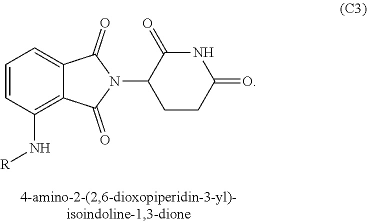

In some embodiments, the ubiquitin ligase recruitment moiety (C) is a small molecule with specificity for the VHL domain of the E3 ligase complex.

In some embodiments, the ubiquitin ligase recruitment moiety (C) is (2S,4R)-1-((R)-2-amino-3,3-dimethylbutanoyl)-4-methyl-N-(4-(4-methylthiazol-5-yl)benzyl)pyrrolidine-2-carboxamide

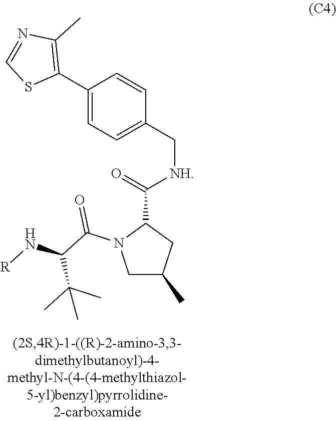

In some embodiments, the ubiquitin ligase recruitment moiety (C) is a small molecule with specificity for the MDM2 domain of the E3 ligase complex.

In some embodiments, the ubiquitin ligase recruitment moiety (C) is (2R,3S,4R,5S)—N-(4-carbamoyl-2-methoxyphenyl)-3-(3-chloro-2-fluorophenyl)-4-(3-chloro-5-fluorophenyl)-4-ethynyl-5-neopentylpyrrolidine-2-carboxamide

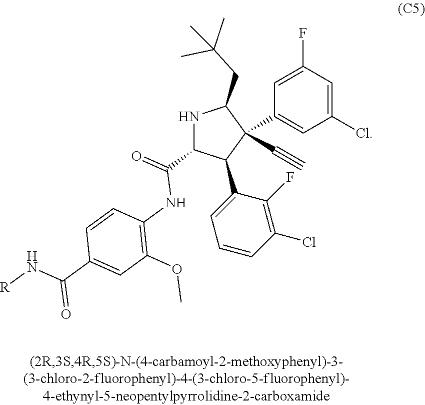

In some embodiments, the ubiquitin ligase recruitment moiety (C) is a small molecule with specificity for the CIAP domain of the E3 ligase complex.

In some embodiments, the ubiquitin ligase recruitment moiety (C) is (S)—N—((S)-2-((S)-2-(4-benzoylthiazol-2-yl)pyrrolidin-1-yl)-1-cyclohexyl-2-oxoethyl)-2-(methylamino)propanamide

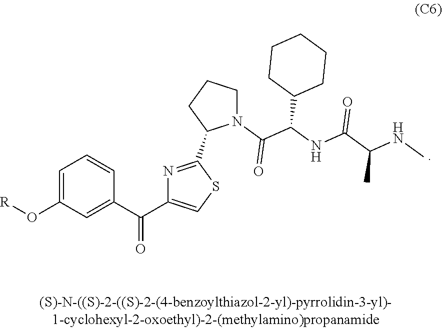

In some embodiments, the ubiquitin ligase recruitment moiety (C) is ((2S,3R)-3-amino-2-hydroxy-4-phenylbutanoyl)leucine

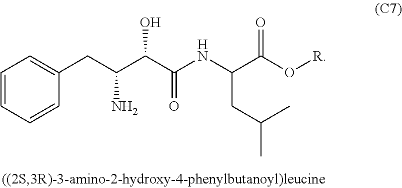

In some embodiments, the ubiquitin ligase recruitment moiety (C) is (S)-2-((S)-1-((S)-2-cyclohexyl-2-((S)-2-(methylamino)propanamido)acetyl)pyrrolidine-2-carboxamido)-3,3-diphenylpropanoic acid

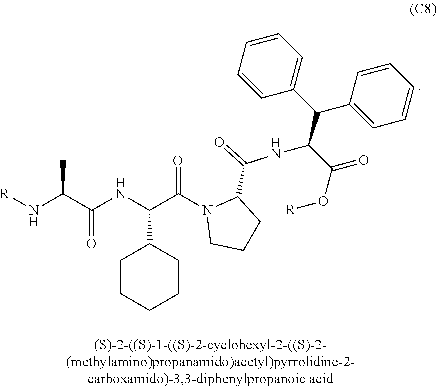

In some embodiments, D is an amino acid or a derivative thereof.

In some embodiments, wherein D is lysine.

In some embodiments, D is azido lysine or cysteine.

In some embodiments, L1, L2, and L3 are the same or different.

In some embodiments, L1, L2, and L3 are each independently a polyethylene glycol (PEG), an amino acid, a hydrocarbon with terminal carboxylic acid and amine groups, or a combination thereof.

In some embodiments, the amino acid is a canonical or nonstandard amino acid.

In some embodiments, the hydrocarbon with terminal carboxylic acid and amine groups is 5-amino pentanoic acid, 10-amino decanoic acid, or 18-amino octadecanoic acid.

In some embodiments, L1, L2, and L3 are each independently a polyethylene glycol (PEG).

In some embodiments, L1 is PEG2, PEG4, PEG8, or PEG12.

In some embodiments, L1 is 2×GGGGS, 4×GGGGS (SEQ ID NO: 11), or 8×GGGGS (SEQ ID NO: 12).

In some embodiments, L2 is PEG2, PEG4, PEG8, or PEG12.

In some embodiments, L2 is 2×GGGGS, 4×GGGGS (SEQ ID NO: 11), or 8×GGGGS (SEQ ID NO: 12).

In some embodiments, L3 is PEG1, PEG2, PEG3, PEG4, or PEG6.

In some embodiments, L3 is G, GG, GS, GGS, GGGS (SEQ ID NO: 9), GGGGS (SEQ ID NO: 10), 2×GGGGS, 4×GGGGS (SEQ ID NO: 11), or 8×GGGGS (SEQ ID NO: 12).

In some embodiments, the compound is represented by the structure of Formula (II):

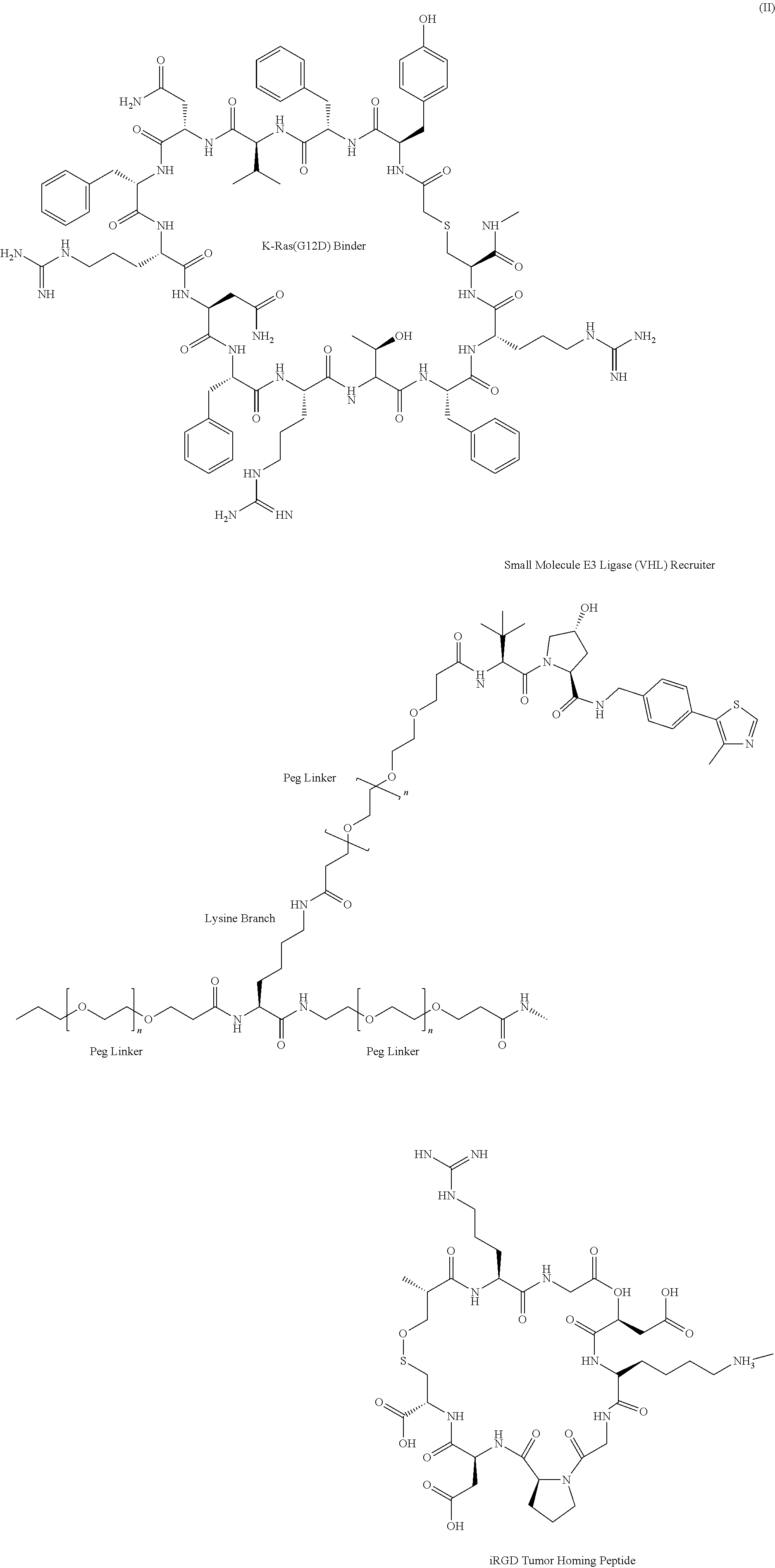

In some embodiments, the compound is represented by the structure of Formula (IV) (also referred to as BXL_2023.001 herein):

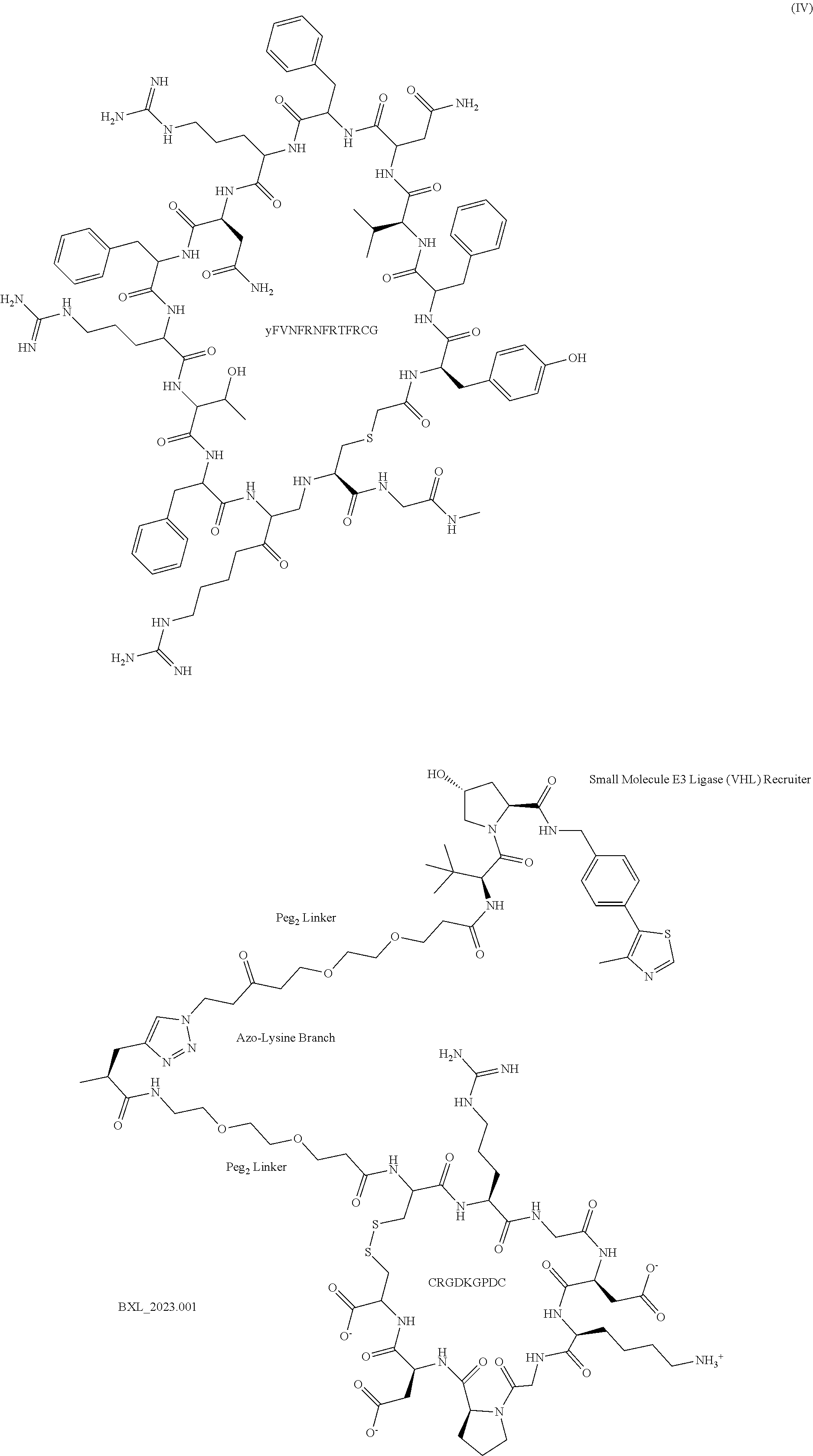

In some embodiments, the peptide sequence of the active cellular targeting moiety (A) contains the cyclic peptide iRGD motif comprising the nine amino acid sequence Cys-Arg-Gly-Asp-Lys-Gly-Pro-Asp-Cys (SEQ ID NO: 1), the ubiquitin ligase recruitment moiety (C) is (2S,4R)-1-((R)-2-amino-3,3-dimethylbutanoyl)-4-methyl-N-(4-(4-methylthiazol-5-yl)benzyl)pyrrolidine-2-carboxamide (C4) and the protein-binding moiety is a small molecule protein recruiter.

In certain embodiments, the small molecule protein recruiter is a bromodomain (BRD4) inhibitor. In some embodiments, the bromodomain (BRD2, BRD3, and BRD4) inhibitor is OTX015. In certain embodiments, the bromodomain (BRD2, BRD3, and BRD4 inhibitor) inhibitor is Molibresib (I-BET762). In some embodiments the bromodomain (BRD4) inhibitor is JQ1 or TEN-010 (JQ2). In embodiments wherein the bromodomain (BRD2, BRD3, and BRD4) inhibitor is OTX015, the compound is represented by the structure of Formula (III)

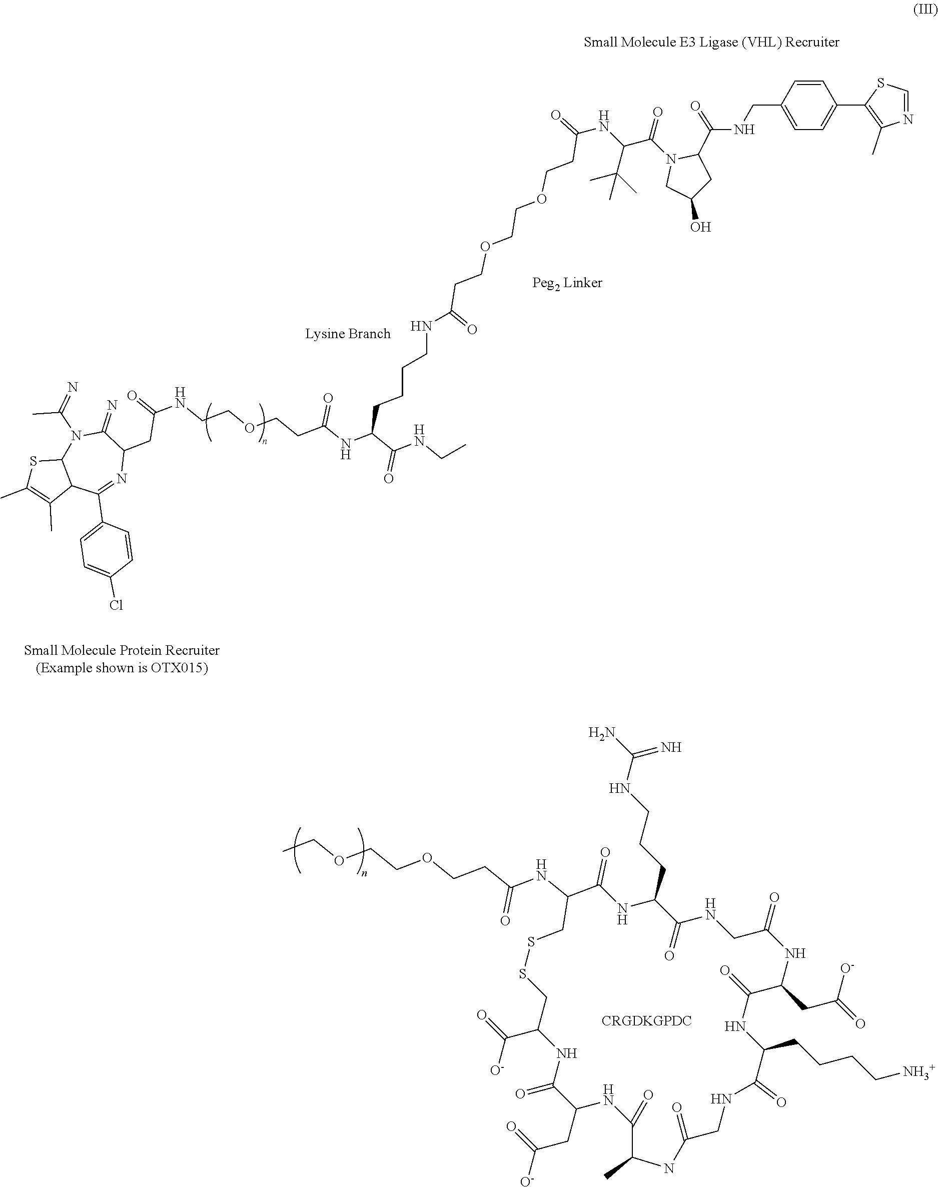

In some embodiments, the compound is capable of degrading a target protein in a cell. In some embodiments, the target protein is a KRAS mutant.

In another aspect, the present invention provides a method for treating a disease or disorder characterized by the overexpression of a specific intracellular protein, comprising administering to a subject in need thereof a therapeutically effective amount of the compound of the invention as described anywhere herein.

In some embodiments of said method, the intracellular protein is a KRAS mutant. In some embodiments of said method, a therapeutically effective amount of the compound of Formula (II) is administered to the subject. In certain embodiments of the method, a therapeutically effective amount of the compound of Formula (III) is administered to the subject.

In some embodiments of the method of the invention, the disease or disorder is selected from immune and inflammatory diseases, infectious diseases, cardiovascular diseases, endocrine disorders, and cancers. In some embodiments of said method, the disease or disorder is pancreatic cancer.

In some embodiments of the method of the invention, the disease or disorder is pancreatic cancer. In some embodiments, the disease or disorder is resistant to traditional therapeutic regimens.

In some embodiments, the compound of the invention further conjugates a fatty diacid, e.g., a C16, to a residue on the peptide of the compound to increase plasma half-life.

In some embodiments, the compound of the invention can be prepared by the methods known in the art. In some embodiments, the compound of the invention is provided as shown in Example 1.

In some embodiments, the compound or conjugate of the invention as described herein is in a pharmaceutically acceptable salt form. Accordingly, it will be appreciated that salt forms of the compound or conjugate of the present invention as described herein are within the scope of this invention.

The term “pharmaceutically acceptable salt” as used herein, in some embodiments, refers to those salts that are safe and effective for pharmaceutical use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, D,L-tartrate, L-tartarate, D-tartarate, pantothenate, bitartrate, ascorbate, succinate, hemisuccinate, maleate, gentisinate, gentisate, fumarate, gluconate, glucaronate, glycolate, saccharate, formate, besylate, benzoate, glutamate, malate, methanesulfonate, ethanesulfonate, benzensulfonate, p-toluenesulfonate, oxalate, tosylate, naphtalen-2-sulfate, or pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

In another aspect, the present invention provides a pharmaceutical composition comprising a mPROTAC conjugate or a compound of the invention as described herein, or a pharmaceutically acceptable salt thereof, in combination with one or more pharmaceutically acceptable carriers.

The term “pharmaceutically acceptable carrier” is art-recognized and refers to a pharmaceutically acceptable material, composition, or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The route of administration of the compounds or conjugates or pharmaceutical compositions of the invention as described herein may be any of those commonly known in the art. For example, the administration can be by any appropriate mode, including orally, parenterally, intravenously, intramuscularly, intraperitoneally, transdermally, intranasally, or by direct infusion with a catheter. The dosage and frequency of administration will depend on the condition of the patient, concurrent administration of other drugs, and other parameters to be taken into account by the clinician.

A proteolysis-targeting chimera (PROTAC) is classically composed of two key components linked together by a single linking group. The current invention dramatically expands the notion of PROTACs by adding multifunctionality (mPROTAC) with cellular targeting moieties. This approach allows for distinct moieties to allow for specific control of each of the essential elements: cell binding, internalization, target interaction, and target modification.

The mPROTAC conjugate of the present invention can be used in treatment for various diseases such as cancer where a tumor-homing peptide is specifically designed to selectively bind to unique receptors that are overexpressed on the surface of various diseased cells. This facet of the mPROTAC conjugate facilitates the accumulation of the therapeutic molecule specifically at the tumor site, reducing systemic exposure and potential off-target effects.

The second component of the mPROTAC conjugate, the target-interacting peptide, is designed to engage with a previously challenging intracellular target protein implicated in cellular homeostasis. Once inside the diseased cell, this element of the mPROTAC conjugate binds to the target protein, initiating its degradation via the proteasomal pathway.

By utilizing the cell's own ubiquitin-proteasome system, the mPROTAC of the invention triggers the degradation of the target protein, effectively disrupting the intracellular processes essential for the survival and proliferation of diseased cells. Similar to previous PROTAC therapies, this dual targeting and degradation approach holds immense therapeutic potential for treating various types of cancer, including those resistant to existing therapies.

The mPROTAC conjugate of the present invention provides a significant advancement in pharmacotherapy, offering a potent, selective, and potentially safer alternative to conventional drug treatments. The immediate uses include chemotherapy, heart failure, and infectious diseases.

But also for any disease process where modification of protein populations would have a therapeutic effect.

The invention presents a unique PROTAC conjugate capable of selective delivery and targeted therapeutic effect on cancer cells. This innovative design offers a promising new approach to cancer treatment, combining targeted delivery, protein interaction specificity, and induced proteasomal degradation.

In another aspect, the present application provides a method for treating a disease or disorder characterized by the overexpression of a specific intracellular protein, comprising administering to a subject in need thereof a therapeutically effective amount of a compound of the invention as described herein. In some embodiments, the intracellular protein is a KRAS mutant.

In some embodiments, disease or disorder is selected from immune and inflammatory diseases, infectious diseases, cardiovascular diseases, endocrine disorders, and cancers. In some embodiments, the disease or disorder is pancreatic cancer. In some embodiments, the disease or disorder is resistant to traditional therapeutic regimens.

As used herein, in some embodiments, the term “multifunctional proteolysis-targeting chimera” and the term “multifunctional proteolysis-targeting chimera conjugate” are used interchangeably.

In some embodiments, the terms “compound” and “conjugate” as described herein are used interchangeably.

The present invention extends the class of heterobifunctional compounds known as Proteolysis-Targeting Chimeras (PROTACs) to enable a more significant number of active moieties through a more significant number of linkages. This novel therapeutic approach utilizes a multifunctional compound comprising a homing molecule—to selectively bind to receptors of the desired cellular target—conjugated with a molecule constructed to interact with a previously intractable intracellular target and an element(s) that activates the cellular proteolytic pathways.

Conjugation of each moiety occurs through a set of linker molecules whose chemistry allows for distinct functionality and the optimization of intracellular target degradation resulting in a therapeutic response. This then provides a potential treatment by modulation of protein populations in various diseases such as immune and inflammatory diseases, infections processes, degenerative cardiovascular diseases, endocrine disorders, and various cancers that are otherwise resistant to traditional therapeutic regimens.

### EXAMPLES

### Example 1: Synthesis of the Compound of the Invention

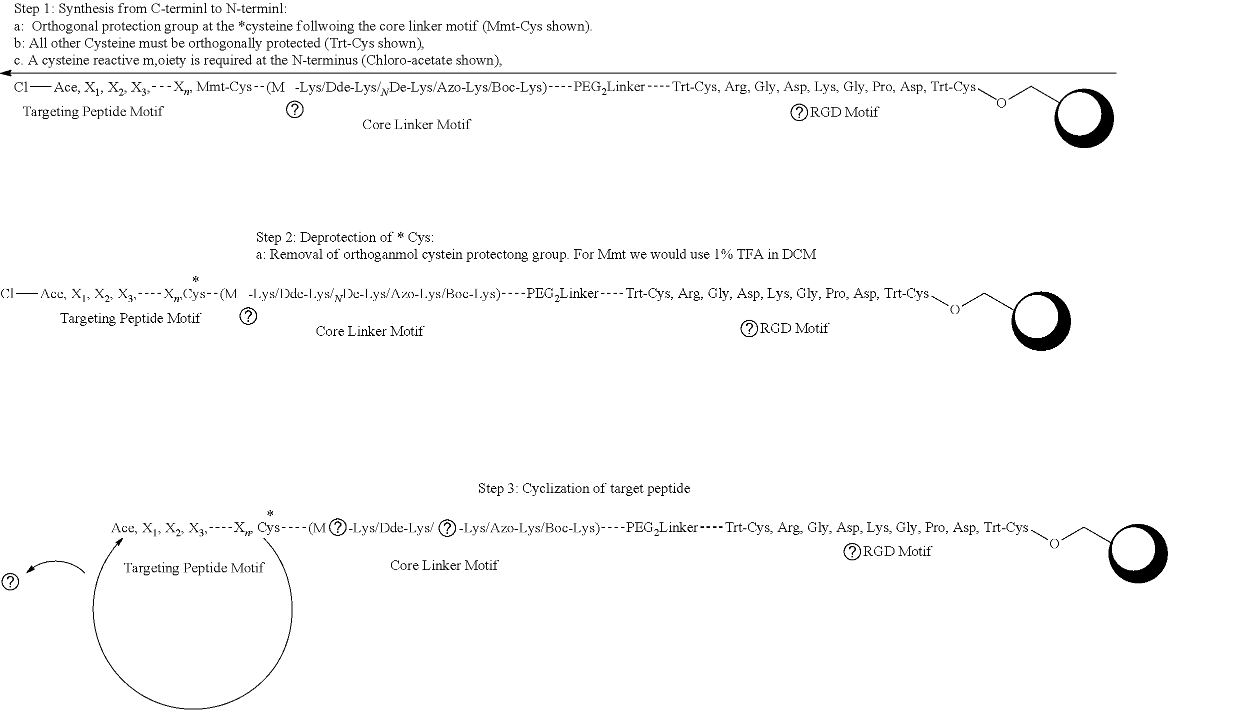

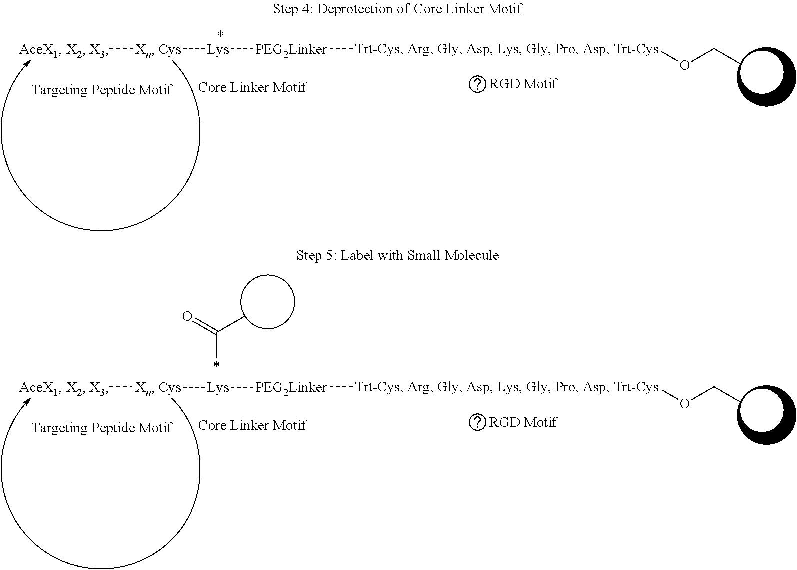

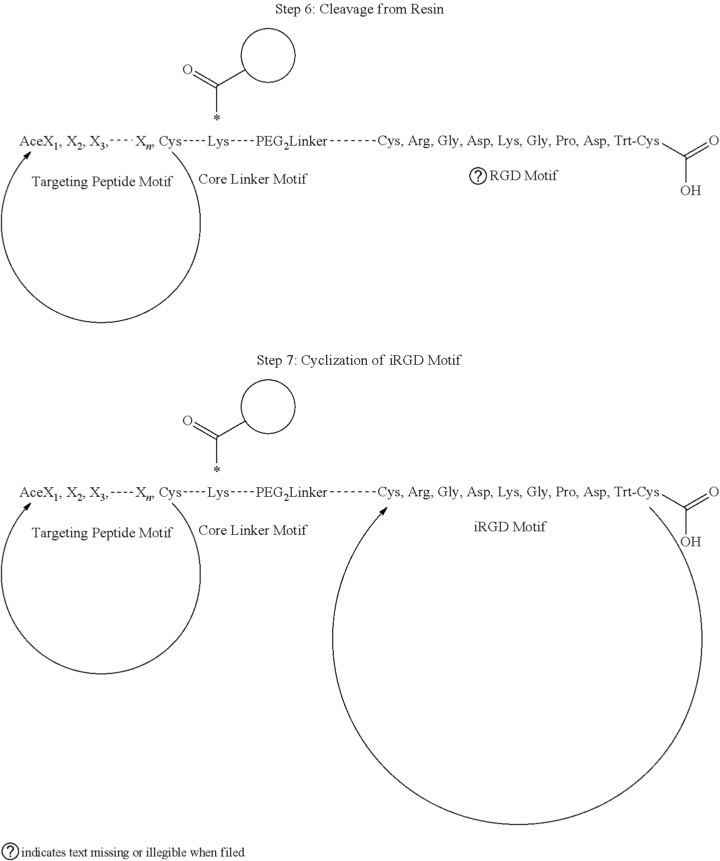

### Example 2: A Cell Viability Assay for a Compound of the Invention Shows Specificity for a Tumor Cell Line

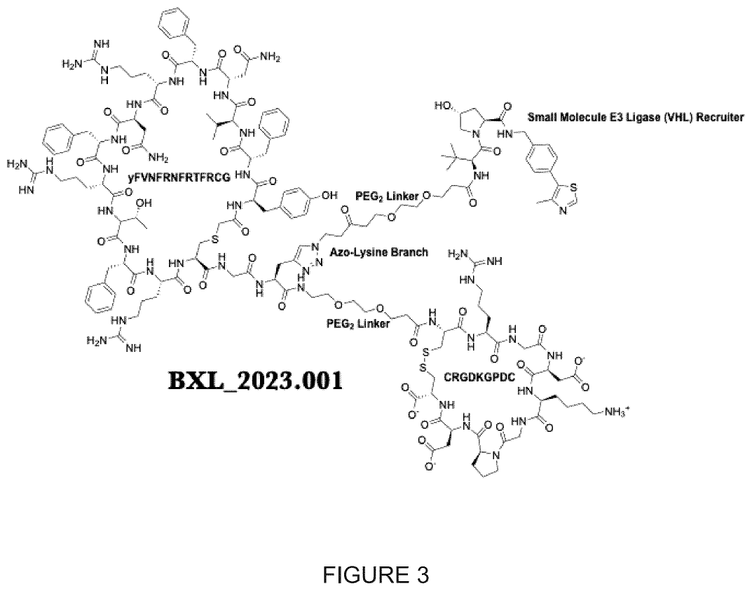

In this example, a cell viability assay was performed using the compound BXL_2023.001 (depicted in FIG. 3) for the pancreatic cancer cell line ASPC-1 (with the HEK-293 cell line as a control).

The cell viability assay was performed according to standard methods. Briefly, both ASPC-1 and HEK293 cells were cultured under standard conditions until ˜80% confluent, and similar passage numbers were maintained for both cell lines to minimize variability.

The cells were detached and seeded into 96-well plates at a density ensuring sub-confluence throughout the experiment. The cells were then allowed to attach overnight. The cells were treated with varying concentrations of the compound the next day and incubated. Untreated cells were included as controls.

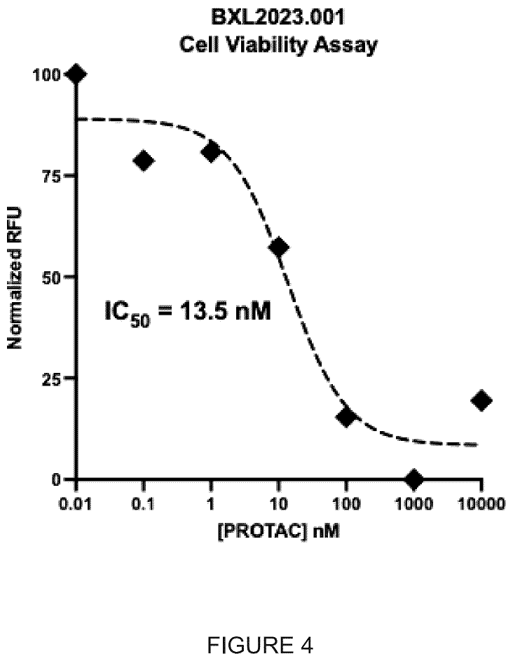

After treatment with the compound for the specified time, the cells were detached, and the cell suspension was mixed with 0.4% trypan blue solution in a 1:1 ratio. The mix was loaded onto a disposable counting slide. The cells were counted with an automatic counter, noting viable and non-viable cells. Cell viability was calculated using the formula: Cell viability (%)=(Number of viable cells/Total cells (viable+non-viable))×100. FIG. 4 is a plot of cell viability as a function of BXL_2023.001 concentration, which shows the specificity of BXL_2023.001 for pancreatic cancer cell line ASPC-1.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound comprising
(a) two or more active moieties,
(b) a central molecule, and
(c) two or more linking groups;
wherein each of the two or more active moieties is independently linked to the central molecule through a linking group;
wherein the active moieties comprise a variety of functions that target extracellular elements, intracellular elements, and intracellular processes;
wherein the active moieties are synergistic; and
wherein the active moieties have a molecular weight of from 100 Da to 10⁶ Da.

2. The compound according to claim 1, wherein the compound is represented by the structure of Formula (I) 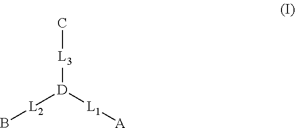
wherein
A is a cellular targeting moiety;
B is a protein-binding moiety;
C is a ubiquitin ligase recruitment moiety;
D is a central linking moiety; and
each of L1, L2, and L3 is independently a linking group.

3. The compound according to claim 2, wherein the active cellular targeting moiety (A) is a linear peptide or a cyclic peptide.

4. The compound according to claim 2, wherein the active cellular targeting moiety (A) targets αVβ3 and αVβ5 integrin receptors, which may be overexpressed on tumor cells followed by neuropilin-1 receptor-mediated cellular uptake.

5. The compound according to claim 4, wherein the peptide sequence of the active cellular targeting moiety (A) contains an RGD peptide motif comprising the three amino acids Arg-Gly-Asp, wherein the peptide sequence comprises the structure of Formula (A1) at neutral pH, 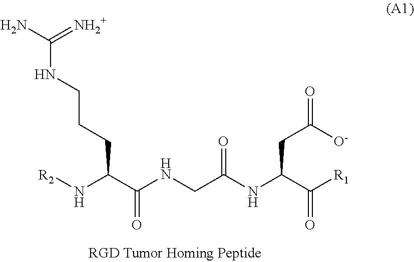
wherein
R₁ is a carboxylate OH or a carboxamide NH₂ and
R₂ is the remaining structure of the compound of Formula (I).

6. (canceled)

7. The compound according to claim 2, wherein the peptide sequence of the active cellular targeting moiety (A) contains a cyclic peptide iRGD motif comprising anine amino acid sequence selected from the group consisting of Cys-Arg-Gly-Asp-Lys-Gly-Pro-Asp-Cys (SEQ ID NO: 1), Cys-Arg-Gly-Asp-Arg-Gly-Pro-Asp-Cys, Cys-Arg-Gly-Asp-Arg-Gly-Pro-Asp-Cys, Cys-Arg-Gly-Asp-Lys-Gly-Pro-Glu-Cys, and Cys-Arg-Gly-Asp-Arg-Gly-Pro-Glu-Cys.

8.-10. (canceled)

11. The compound according to claim 7, wherein the peptide is cyclized via a disulfide bond between cysteine at residue position 1 and cysteine at residue position 9, wherein the peptide sequence comprises the structure of Formula (A2) at neutral pH 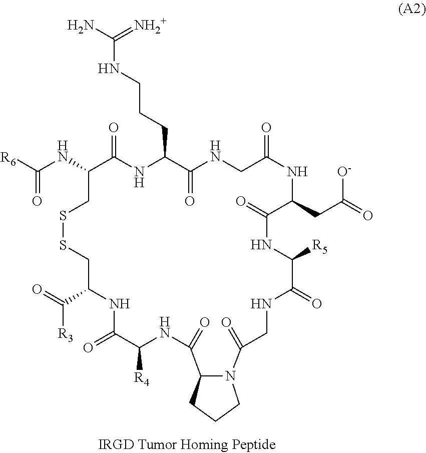
wherein
R₃ is a carboxylate OH or a carboxamide NH₂;
R₄ is an aspartic acid or a derivative thereof, or a glutamic acid or a derivative thereof:
R₅ is a lysine or a derivative thereof, or an arginine or a derivative thereof; and
R₆ is the remaining structure of the compound of Formula (I).

12. (canceled)

13. The compound according to claim 2, wherein the peptide sequence of the active cellular targeting moiety (A) contains an NGR motif comprising the three amino acids Asn-Gly-Arg, wherein the peptide sequence comprises the structure of Formula (A3) at neutral pH 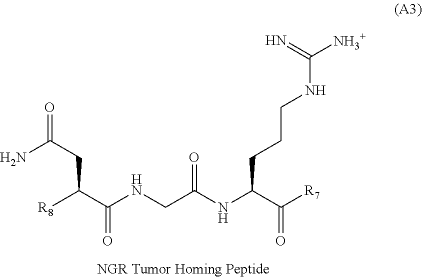
wherein
R₇ is a carboxylate OH or a carboxamide NH₂; and
R₈ is the remaining structure of the compound of Formula (I).

14. (canceled)

15. The compound according to claim 13, wherein the peptide sequence contains the NGR motif comprising the five amino acids Cys-Asn-Gly-Arg-Cys (SEQ ID NO: 2).

16. The compound according to claim 15, wherein the peptide is cyclized with a disulfide bond between cysteine at residue position 1 and cysteine at residue position 5.

17.-18. (canceled)

19. The compound according to claim 2, wherein the peptide sequence of the active cellular targeting moiety (A) contains acyclic peptide comprising the nine amino acid sequence Cys-Gly-Asn-Lys-Arg-Thr-Arg-Gly-Cys (SEQ ID NO: 3).

20. The compound according to claim 19, wherein the peptide is cyclized with a disulfide bond between cysteine at residue position 1 and cysteine at residue position 9.

21.-22. (canceled)

23. The compound according to claim 2, wherein the peptide sequence of the active cellular targeting moiety (A) is selected from the group consisting of a sequence comprising the 29 amino acids Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Lys-Ala-Pro-Ala-Lys-Lys (SEQ ID NO: 4), a sequence comprising the amino acids Cys-Arg-Glu-Lys-Ala (SEQ ID NO: 5), a sequence comprising the amino acids Lys-Glu-Thr-Trp-Trp-Glu-Thr-Trp-Trp-Thr-Glu-Trp-Ser-Gln-Pro-Lys-Lys-Lys-Arg-Lys-Val (SEQ ID NO: 6), and a sequence comprising the amino acids Lys-Glu-Thr-Trp-Trp-Glu-Thr-Trp-Trp-Thr-Glu-Trp-Ser-Gln-Pro-Lys-Lys-Lys-Arg-Lys-Val-cysteamine (SEQ ID NO: 7).

24.-33. (canceled)

34. The compound according to claim 2, wherein the protein-binding moiety (B) is a linear peptide or a cyclic peptide.

35. The compound according to claim 34, wherein the protein-binding moiety (B) is a cyclic peptide with the sequence dTyr-Phe-Val-Asn-Phe-Arg-Asn-Phe-Arg-Thr-Phe-Arg-Cys-Gly (SEQ ID NO: 8), wherein dTyr represents the D-isomer of tyrosine.

36. The compound according to claim 35, wherein the peptide is cyclized with a thioether bond between dTyr at residue position 1 and residue position 13.

37. (canceled)

38. The compound according to claim 2, wherein the ubiquitin ligase recruitment moiety (C) is a small molecule with specificity for the CRBN domain of the E3 ligase complex, the VHL domain of the E3 ligase complex, the MDM2 domain of the E3 ligase complex, or the CIAP domain of the E3 ligase complex.

39. The compound according to claim 38, wherein the ubiquitin ligase recruitment moiety (C) is selected from the group consisting of 2-(2,6-dioxopiperidin-3-yl)-4-hydroxyisoindoline-1,3-dione 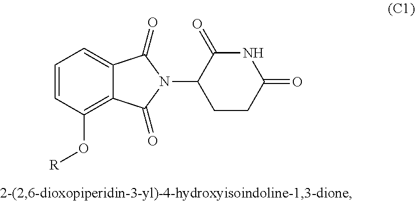
3-(4-amino-1-oxoisoindolin-2-yl)piperidine-2,6-dione 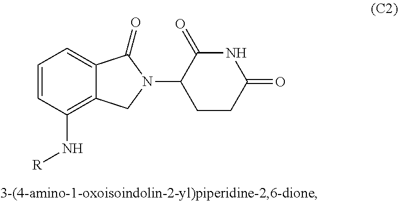
4-amino-2-(2,6-dioxopiperidin-3-yl)isoindoline-1,3-dione 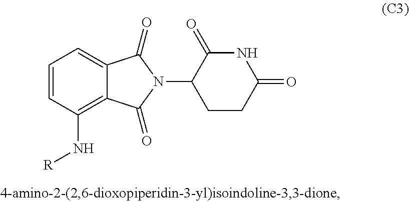
(2S,4R)-1-((R)-2-amino-3,3-dimethylbutanoyl)-4-methyl-N-(4-(4-methylthiazol-5-yl)benzyl)pyrrolidine-2-carboxamide 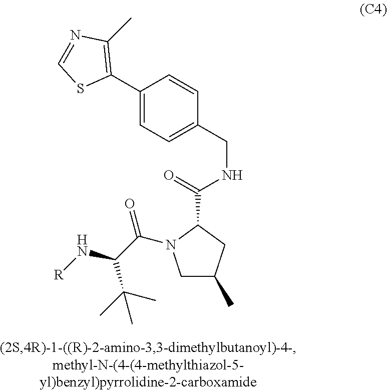
(2R,3S,4R,5S)—N-(4-carbamoyl-2-methoxyphenyl)-3-(3-chloro-2-fluorophenyl)-4-(3-chloro-5-fluorophenyl)-4-ethynyl-5-neopentylpyrrolidine-2-carboxamide 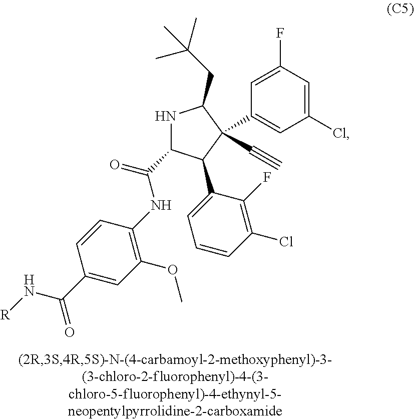
(S)—N—((S)-2-((S)-2-(4-benzoylthiazol-2-yl)pyrrolidin-1-yl)-1-cyclohexyl-2-oxoethyl)-2-(methylamino)propanamide 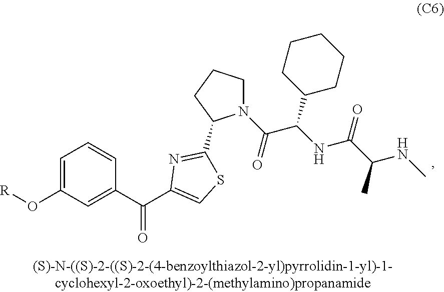
((2S,3R)-3-amino-2-hydroxy-4-phenylbutanoyl)leucine 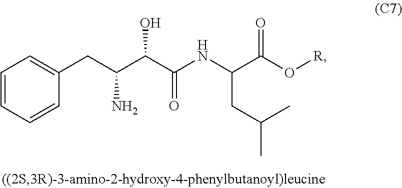
(S)-2-((S)-1-((S)-2-cyclohexyl-2-((S)-2-(methylamino)propanamido)acetyl)pyrrolidine-2-carboxamido)-3,3-diphenylpropanoic acid

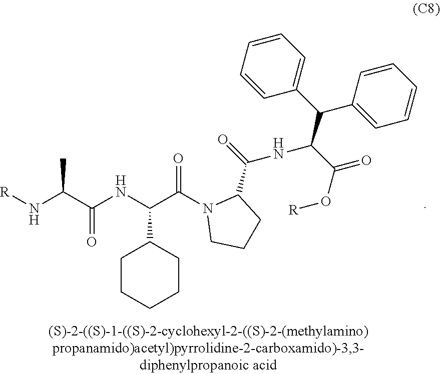

40.-49. (canceled)

50. The compound according to claim 2, wherein D is an amino acid or a derivative thereof.

51. The compound according to claim 50, wherein D is lysine, azido lysine, or cysteine.

52. (canceled)

53. The compound according to claim 50, wherein L1, L2, and L3 are the same or different.

54. The compound according to claim 2, wherein L1, L2, and L3 are each independently a polyethylene glycol (PEG), an amino acid, a hydrocarbon with terminal carboxylic acid and amine groups, or a combination thereof.

55. The compound according to claim 54, wherein the amino acid is a canonical or nonstandard amino acid.

56. The compound according to claim 54, wherein the hydrocarbon with terminal carboxylic acid and amine groups is 5-amino pentanoic acid, 10-amino decanoic acid, or 18-amino octadecanoic acid.

57. The compound according to claim 2, wherein L1, L2, and L3 are each independently a polyethylene glycol (PEG).

58. The compound according to claim 2, wherein L1 and/or L2 and/or L3 is PEG2, PEG4, PEG8, PEG12, 2×GGGGS, 4×GGGGS (SEQ ID NO: 11), or 8×GGGGS (SEQ ID NO: 12).

59.-63. (canceled)

64. The compound according to claim 1, wherein said compound is represented by the structure of Formula (II)

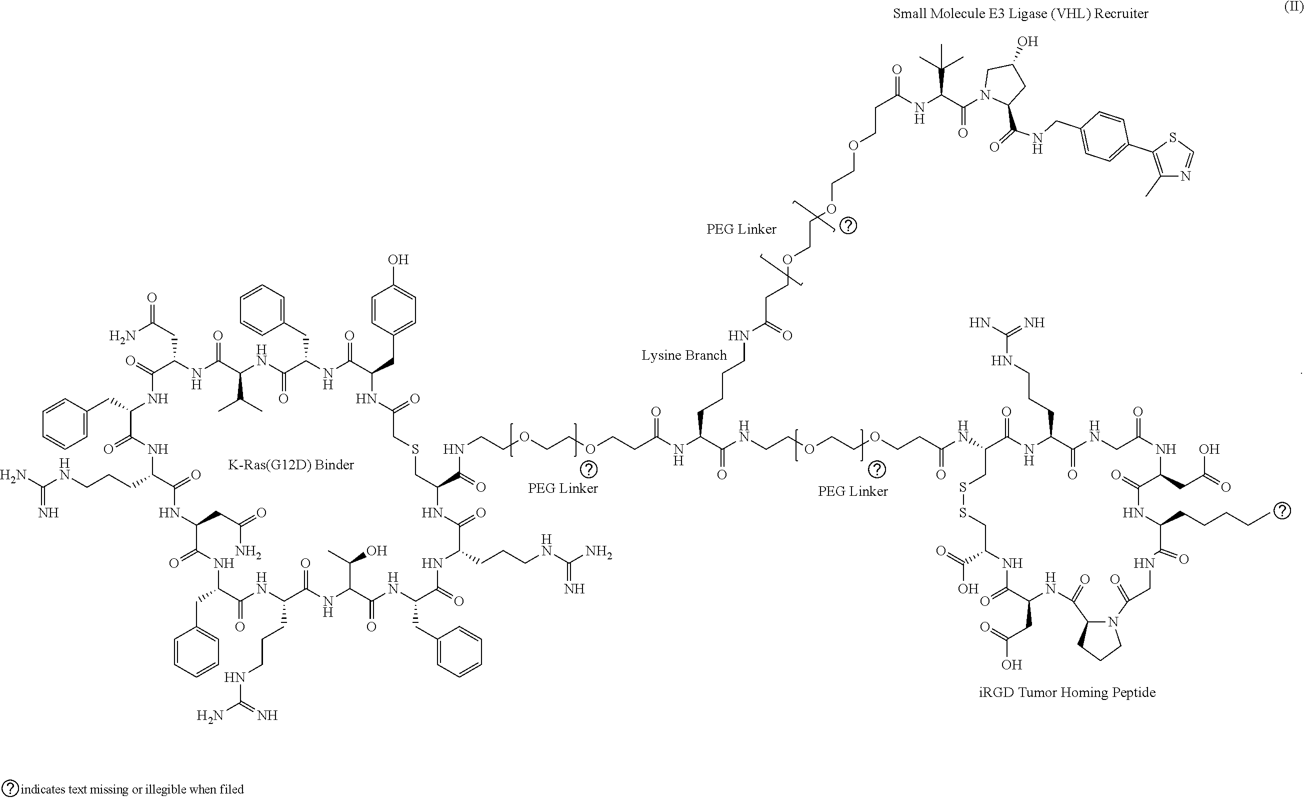

65. The compound according to claim 1, wherein said compound is represented by the structure of Formula (II)

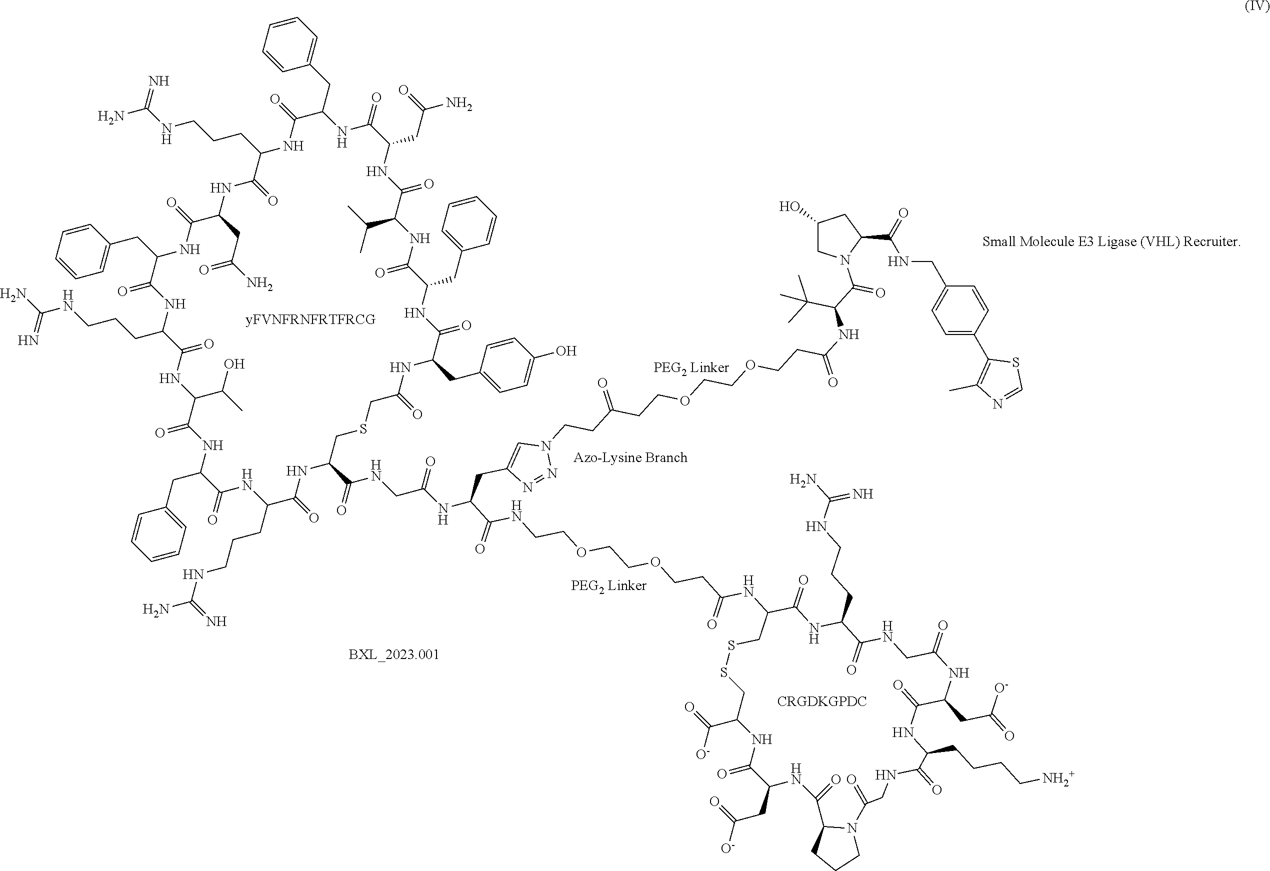

66. The compound according to claim 1, wherein the peptide sequence of the active cellular targeting moiety (A) contains a cyclic peptide iRGD motif comprising the nine amino acid sequence Cys-Arg-Gly-Asp-Lys-Gly-Pro-Asp-Cys (SEQ ID NO: 1), the ubiquitin ligase recruitment moiety (C) is (2S,4R)-1-((R)-2-amino-3,3-dimethylbutanoyl)-4-methyl-N-(4-(4-methylthiazol-5-yl)benzyl)pyrrolidine-2-carboxamide (C4), and the protein-binding moiety is a small molecule protein recruiter.

67. The compound according to claim 66, wherein the small molecule protein recruiter is a bromodomain (BRD2, BRD3, and BRD4) inhibitor.

68. The compound according to claim 67, wherein the bromodomain (BRD2, BRD3, and BRD4) inhibitor is OTX015 and said compound is represented by the structure of Formula (III)

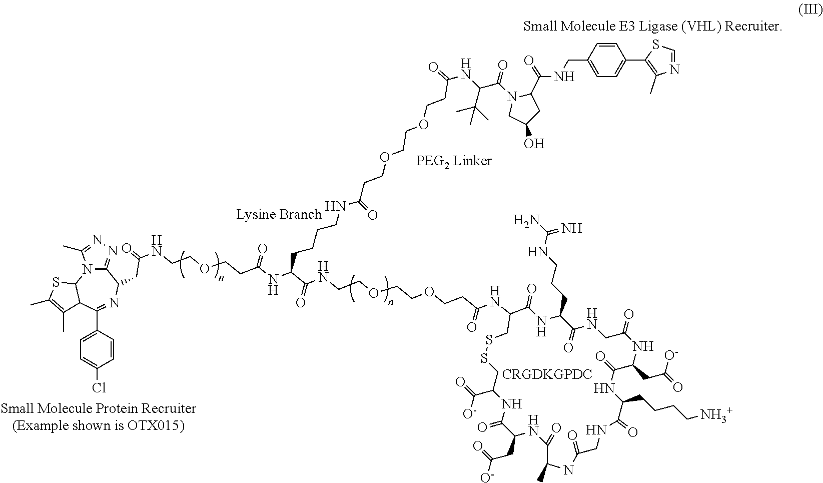

69. The compound according to claim 1, wherein the compound is capable of degrading a target protein in a cell.

70. The compound according to claim 69, wherein the target protein is a KRAS mutant.

71. A method for treating a disease or disorder characterized by the overexpression of a specific intracellular protein, comprising administering to a subject in need thereof a therapeutically effective amount of the compound according to claim 1.

72. The compound according to claim 71, wherein the intracellular protein is a KRAS mutant.

73. The method according to claim 72, wherein said disease or disorder is selected from immune and inflammatory diseases, infectious diseases, cardiovascular diseases, endocrine disorders, and cancers.

74. The method according to claim 73, wherein said disease or disorder is pancreatic cancer.

75. The method according to claim 73, wherein said disease or disorder is resistant to traditional therapeutic regimens.


SEQUENCE LISTING (12 sequences)

SEQ ID NO: 1 - protein, 9 aa, synthetic construct
  REGION 1..9: note = Description of sequence: Active cellular targeting moiety
  VARIANT 5: note = arginine
  VARIANT 8: note = glutamic acid
  DISULFID 1..9: note = cysteine-cysteine bond
  REGION 1..9: note = cyclic
CRGDKGPDC

SEQ ID NO: 2 - protein, 5 aa, synthetic construct
  REGION 1..5: note = Description of sequence: Active cellular targeting moiety
  DISULFID 1..5: note = cysteine-cysteine bond
  REGION 1..5: note = cyclic
CNGRC

SEQ ID NO: 3 - protein, 9 aa, synthetic construct
  REGION 1..9: note = Description of sequence: Active cellular targeting moiety
  DISULFID 1..9: note = cysteine-cysteine bond
  REGION 1..9: note = cyclic
CGNKRTRGC

SEQ ID NO: 4 - protein, 29 aa, synthetic construct
  REGION 1..29: note = Description of sequence: Active cellular targeting moiety
KDEPQRRSARLSAKPAPPKPEPKKAPAKK

SEQ ID NO: 5 - protein, 5 aa, synthetic construct
  REGION 1..5: note = Description of sequence: Active cellular targeting moiety
CREKA

SEQ ID NO: 6 - protein, 21 aa, synthetic construct
  REGION 1..21: note = Description of sequence: Active cellular targeting moiety
KETWWETWWTEWSQPKKKRKV

SEQ ID NO: 7 - protein, 22 aa, synthetic construct
  REGION 1..22: note = Description of sequence: Active cellular targeting moiety
  SITE 22: note = X is cysteamine
KETWWETWWTEWSQPKKKRKVX

SEQ ID NO: 8 - protein, 14 aa, synthetic construct
  REGION 1..14: note = Description of sequence: Active cellular targeting moiety
  SITE 1: note = d-tyrosine
  CROSSLNK 1..14: note = Crosslink is between d-tyrosine N-terminal amine and cysteine side-chain thiol
  REGION 1..14: note = cyclic
YFVNFRNFRTFRCG

SEQ ID NO: 9 - protein, 4 aa, synthetic construct
  REGION 1..4: note = Description of sequence: Linker
GGGS

SEQ ID NO: 10 - protein, 5 aa, synthetic construct
  REGION 1..5: note = Description of sequence: Linker
GGGGS

SEQ ID NO: 11 - protein, 20 aa, synthetic construct
  REGION 1..20: note = Description of sequence: Linker
GGGGSGGGGSGGGGSGGGGS

SEQ ID NO: 12 - protein, 40 aa, synthetic construct
  REGION 1..40: note = Description of sequence: Linker
GGGGSGGGGSGGGGSGGGGSGGGGSGGGGSGGGGSGGGGS